(19) United States
(12) Patent Application Publication
(10) Pub. No.: US 2004/0241675 A1
(43) Pub. Date: Dec. 2, 2004

(54) Method and device for determining and selecting molecule-molecule interactions

(76) Inventors: Arnold Gillner, Roetgen (DE); Elke Bremus-Kobberling, Greveubroich (DE); Stefan Barth, Roetgen (DE); Rainer Fischer, Monschau (DE)

Correspondence Address: NEEDLE & ROSENBERG, P.C., SUITE 1000, 999 PEACHTREE STREET, ATLANTA, GA, 30309-3915, US

(21) Appl. No.: 10/474,602

(22) PCT Filed: Apr. 10, 2002
(86) PCT No.: PCT/EP02/03983

(30) Foreign Application Priority Data
Apr. 10, 2001 (EP) 01108919.0

(51) Int. Cl. C12Q 1/68

(52) U.S. Cl. 435/6

(57) ABSTRACT

The invention relates to biochips for documenting of specific molecule-molecule interactions or cell-molecule interactions, more particularly unknown singular protein interactions. The invention also relates to corresponding methods for selecting and characterizing molecules, especially polypeptides and the complementary binding molecules.

The present invention relates to biochips for the documentation of specific molecule-molecule interactions or cell-molecule interactions, especially of unknown singular protein interactions, and corresponding methods for the selection and characterization of molecules, especially polypeptides and their complementary binding molecules.

### BACKGROUND OF THE INVENTION

Currently, the interactions between proteins can be represented by various techniques. Recently, techniques have been developed which enable the selection of protein libraries on immobilized libraries. Mainly by coupling the DNA/RNA to be expressed to the protein expressed by it in bacteriophages (“phage display”) of by “ribosome display” or profusion technology, the genetic information of the enriched proteins is immediately available. The current screening methods are characterized in that the information relating to the individual members of the immobilized libraries is known and localized on predefined positions on the chips.

Usually, within the scope of the usual systems, one or more clones from the soluble protein library become enriched in higher numbers of copies. Currently, singular unknown clones (in terms of a single molecule) cannot be examined immediately; selected bacteriophages must be usually proliferated before a detailed analysis can be done. If unknown members of a protein library were immobilized, an immediate analysis of the immobilized binding molecule detected by protein interaction would not be possible either currently.

These problems are solved by the biochips employed herein, which thereby allow the use of methods for the high-throughput analysis of molecule-molecule interactions, especially of proteins, on the single molecule level.

### SUMMARY OF THE INVENTION

Thus, the present invention relates to:

(1) a method for the selection of unknown molecules comprising the following steps:

(a) immobilizing molecules of a combinatory molecule library on a support;

(b) attaching complementary singular binding molecules or cells to a particular immobilized molecule;

(c) selective detaching the mutually interacting singular molecules from the support; and

(d) analyzing the material detached in step (c);

(2) a biochip for the selection of unknown single molecules comprising a support as defined under (1) on which a combinatory molecule library can be or has been immobilized, ensuring that precisely one molecule can be or has been taken up on the support per coupling area;

(3) a method for the preparation of a biochip as defined under (2), comprising:

(i) producing coupling areas on a support which are suitable for taking up precisely one molecule; and/or

(ii) immobilizing molecules from a combinatory molecule library on a support; and

(4) a device for the automated measurement of molecular or cellular interactions and their coordinative assignment with a spatial resolution of <100 nm, especially for use in a method as defined under (1) and/or for reading out a biochip as defined under (2).

### DESCRIPTION OF FIGURES

FIG. 1 shows the general set-up of a microchip of the invention.

FIG. 2 shows how the position of the attached molecule or of an attached cell can be determined.

FIG. 3 shows the schematic structure of particle A of the exemplary system of the invention.

FIG. 4 shows the schematic structure of particle B of the exemplary system of the invention.

FIG. 5 shows the schematic structure of interacting particles A+B.

### DETAILED DESCRIPTION OF THE INVENTION

“Support” within the meaning of the present invention comprises support structures of various dimensions including microstructures (chips), but also larger structures, such as glass slides and plates in the decimeter range. Suitable support materials include signal-transmissive materials, especially materials transmissive for light, heat and/or magnetic fields, more preferably light-transmissive materials (i.e., supports of glass, silicone, light-transmissive PVC etc.).

The supports according to the invention (also briefly referred to as “biochips” in the following) are composed of arrays of isolated molecules from combinatory libraries (e.g. proteins, peptides etc.). The size of the areas on which the molecules are fixed (also referred to as “areas” in the following) and the mutual distance of these areas must be selected to ensure that the individual molecules can be identified.

Therefore, the distance and size of the areas depend on the size of the molecules, inter alia. The distance between the areas is preferably within a range of from 10 nm to 100 μm, preferably from 100 nm to 5.0 μm and more preferably from 100 nm to 1,500 nm. The size of the areas is preferably smaller than 50 nm×50 nm, more preferably from 5×5 nm to 20×20 nm (or corresponding areas with a different geometry). This is enabled by:

a) coupling an anchor group reactive towards light (magnetism or heat) (e.g., o-nitrobenzyl ester, pivaloyl or triazene compound) for molecules (e.g., polypeptides) to the chip;

b) for preparing the molecule coupling, adjusting coupling-reactive square (but also rectangular, circular etc.) areas with a size and distance between the areas, as mentioned above;

c) by coupling an individual molecule to each of said coupling-reactive areas.

A method according to the invention for the selection of interacting molecules (two- and more-component systems) comprises the following steps:

a) establishing a first combinatory library, especially a cDNA expression library for binding proteins (for example, antibodies), alternatively a cDNA expression library of a cell, or a synthetic peptide or molecule library, optionally by direct synthesis on the chips;

b) coupling the library to the biochips according to the invention through an anchor group activatable by light (heat or magnetism) (e.g., the photoactive o-nitrobenzyl esters, pivaloyl linkers or triazene compounds as specified below);

c) establishing a second combinatory library, especially a cDNA expression library of a cell, alternatively a cDNA expression library for binding proteins, or a synthetic peptide or molecule library;

d) screening for individual interactions between the immobilized library and the soluble library by “two(or more)-coordinate” laser measuring technology (through the axes X and Y), or alternative measuring techniques;

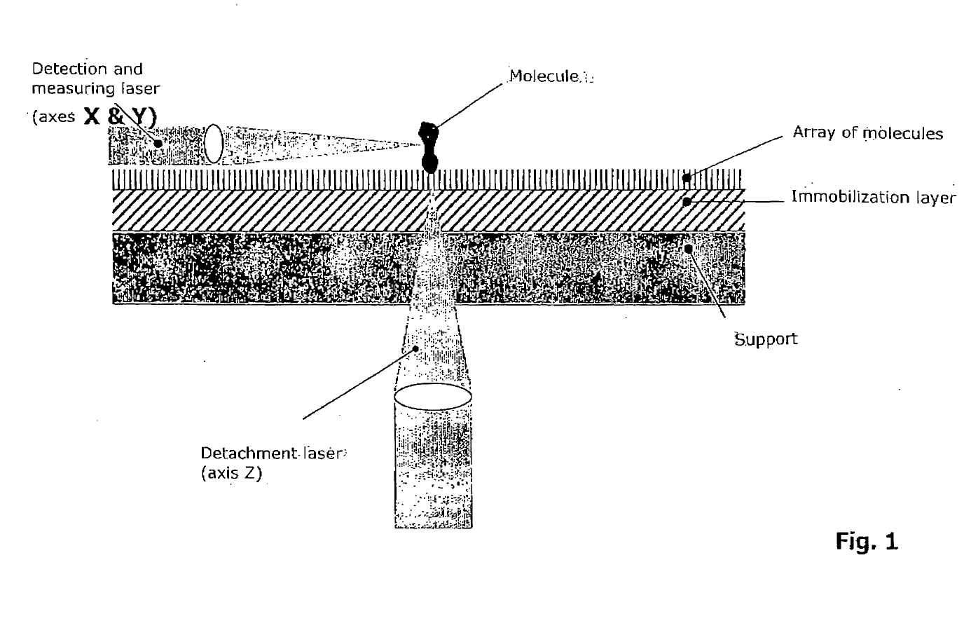

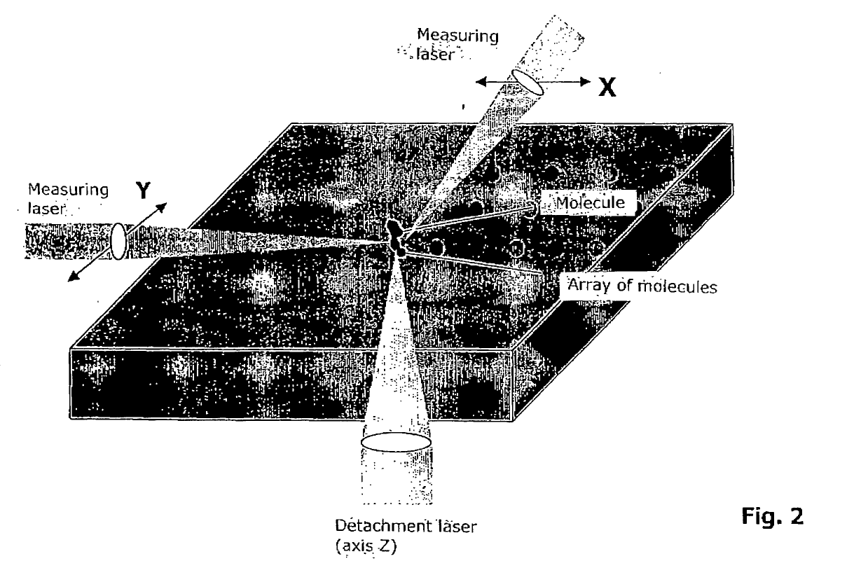

e) selected light-dependent detachment of the interacting molecules from the biochip through a third axis Z (see also FIGS. 1 and 2);

wherein said first library (e.g., a cDNA expression library) may be enriched with complementary binding molecules according to described methods, and said second library may be, e.g., a subtractive cDNA expression library, in particular.

“Complementary binding molecules” include, in particular, polypeptides which specifically interact with defined molecules. A specific interaction means a dissociation constant KD of <10⁻⁶ mol/l, preferably <10⁻⁸ mol/l.

“cDNA expression library for binding proteins” means a great number of substances wherein, in an individual component, the coding region of a nucleic acid is specifically linked with the related expressed binding-active polypeptide. Typical examples thereof include libraries of complementary binding peptides in transformed cells (preferably bacteria and yeasts, more preferably bacteriophages) in which the incorporated nucleic acid is functionally translated and the protein is expressed into the cytosol or is anchored to the surface of the cell as a fusion protein, in particular. Thus, proceeding from the properties of the binding peptide, an analysis of its binding function with the membrane-associated polypeptide can be performed according to the current state of the art, and the correlating nucleic acid can be unambiguously assigned at the same time. This similarly applies to the “ribosome display” or liposome display or profusion technology.

The establishing of cDNA expression libraries for complementary binding peptides in bacteriophages is described, for example, in U.S. Pat. No. 5,969,108, and for display on ribosomes in U.S. Pat. No. 5,643,768.

“cDNA expression libraries of a cell” means a great number of unknown individual peptidic molecules wherein, in an individual component, the coding region of an mRNA transformed into a cDNA is specifically linked with the related expressed polypeptide. Typical examples thereof include cDNA expression libraries, for example, in λ phages or other filamentous bacteriophages, which ensure that the incorporated nucleic acids are functionally translated in bacteria, and after the lysis of the bacteria, the expressed protein is exposed in corresponding plaques or at the surface of compartments (liposomes, cells, bacteria, bacteriophages) which ensure that the translation products of the incorporated nucleic acids are functionally exposed on the outside of the compartments. Thus, these cDNA expression libraries include the image of the expressed proteins of a cell, so that molecule-molecule interactions can be documented by the “two(or more)-coordinate” laser technology according to the invention by combination with the corresponding complementary binding peptide, starting from the properties of interacting polypeptides.

“Synthetic peptide or molecule library” means a great number of peptides or molecules which have been composed by combinatory synthesis, whereby all theoretically combinable molecule compositions become available in an ideal case.

“Immobilization” means that the members of one library are bound to a solid matrix (e.g., immobilization by means of anchoring groups, such as o-nitrobenzyl ester, pivaloyl or triazene compounds, which can be separated particularly easily by a light (heat or magnetic) reaction after the interaction with the second peptide has been documented. In particular, suitable anchoring groups for the immobilization include the following compounds as mentioned in WO 95/31429 and WO 99/67619:

Photolinkers Based on o-nitrobenzyl esters (linker I):

λ_{cleavage}=365 nm

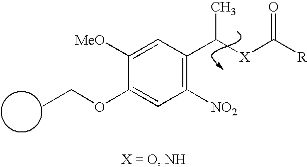

Pivaloyl Linkers (linker II):

λ_{cleavage}≈280 to 340 nm

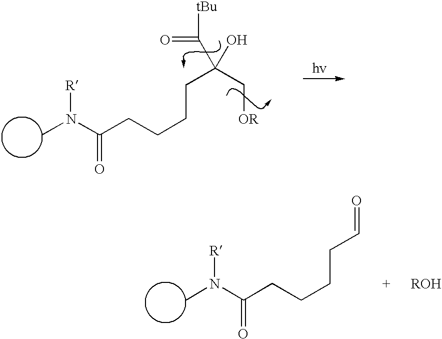

and triazene compounds.

The general structure of the biochips is shown in FIG. 1. The basis of the biochips is formed by a quartz or glass plate or, alternatively, a plastic support, which must be signal-transmissive, however (for light, heat, magnetic fields). Onto this support is coated first an adhesive layer (e.g., of polylysine derivatives) and then an immobilization layer which, on the one hand, adheres to the support and, on the other hand, undergoes a firm (covalent) binding with the individual molecules to be immobilized from the combinatory libraries on the side facing towards the protein. The immobilization layer consists of either chemical molecules or compounds which can be chemically cleaved by light excitation, or molecules or compounds which are separated by thermal action.

After loading with the adhesive layer and photolinker layer, the coupling-active areas, i.e., the desired structures on the support, are generated. Alternatively, the photolinker layer may also be applied after adjustment of the structure of the adhesive layer.

This immobilization layer is loaded with individual molecules of a combinatory library to ensure that one molecule will bind to one site, but not to two neighboring sites. This is achieved by allowing a distance of from 10 nm to 1000 μm (preferably from 100 nm to 5000 nm or from 100 nm to 1500 nm) between the arrays, depending on the maximum size of the individual molecule, i.e., the molecules are isolated according to a predetermined positional grid.

In this form, the thus prepared biochips can be exposed to a second combinatory library. Particular molecules from the soluble library will attach to individual molecules of the immobilized library of the biochip. Now, using a suitable measuring technique, the position of the attached molecule or even an attached cell with respect to the respective protein is determined with a high spatial resolution (FIG. 2). As the measuring techniques, the following methods can be employed:

Fluorescence measurements on a molecule (peptide, protein, conjugated ribosome, conjugated bacteriophage, liposome, labeled particles and cells) previously provided with a fluorescent label;

simulated Raman emissions from the attached molecule (peptide, protein, conjugated ribosome, conjugated bacteriophage, liposome, labeled particles and cells);

magnetic detection of the attachment site through a molecule (peptide, protein, conjugated ribosome, conjugated bacteriophage, liposome, labeled particles and cells) coupled to a magnetic bead;

atomic force microscopy by means of which a local enrichment of a molecule (peptide, protein, conjugated ribosome, conjugated bacteriophage, liposome, labeled particles and cells) can be determined with a high spatial resolution;

wherein the measuring method must be designed in such a way that a spatial resolution of <100 nm can be achieved, if necessary.

After washing the biochip and removing all unattached components and residual molecules, the actual measurement of the molecule interactions is performed at the attachment site (through axes X and Y). After the determination of the attachment site, a laser beam is directed through the biochip using a second scanning system, i.e., through the support from below (axis Z) onto the immobilization layer (e.g., made of the o-nitrobenzyl ester, pivaloyl or triazene compounds) as defined above. The position of this exposure corresponds to the previously determined position of the attached molecule. Subsequently, the immobilization layer is briefly irradiated to cleave the chemical bonds of the photolabile linker or anchoring group of the immobilization layer, or the immobilization layer is detached thermally. Thus, the molecules interacting at this site can be detached again specifically. This means that both the previously immobilized molecule (peptide, protein, conjugated ribosome, conjugated bacteriophage, liposome, labeled particle) and the related soluble molecule (peptide, protein, conjugated ribosome, conjugated bacteriophage) are available for the subsequent analysis.

Through the subsequent analysis of the thus selected interacting molecules, for example, by “matrix-associated laser desorption/ionization” (MALDI) or “electrospray ionization (ESI) with subsequent mass analysis in a time-of-flight (TOF or OTOF), quadrupole (Q), ion trap (IT), Fourier transform (FTMS or FT-ICR) mass spectrometer, “post-source decay” PSD, “in-source decay” (ISD), “surface plasmon resonance” (SPR), multispectral analysis, fluorescence correlation spectroscopy (FCS) or polymerase chain reaction (PCR), the structure of the protein and its DNA can be derived and its DNA optionally replicated.

Independently of the above mentioned analysis and detaching mechanisms, other space-resolving detection methods and activation technologies may also be used.

Exemplary System

Step 1: Synthesis of the Microchips

On a quartz or glass substrate, an adhesion layer of polylysine derivatives is first applied, and then the photolinker layer consisting of linker I (with X=0) and a particle-A-reactive ligand (here: biotin) are coupled thereto. By laser technology, structures such areas having a diameter of <50 nm are generated at a distance of 1.2 μm as the pattern. Since, in the exemplary system presented here, the length of the particle to be immobilized (particle A) is about 1 μm and it is to be prevented that one particle A binds to two coupling areas, this minimum distance between the coupling areas must be ensured.

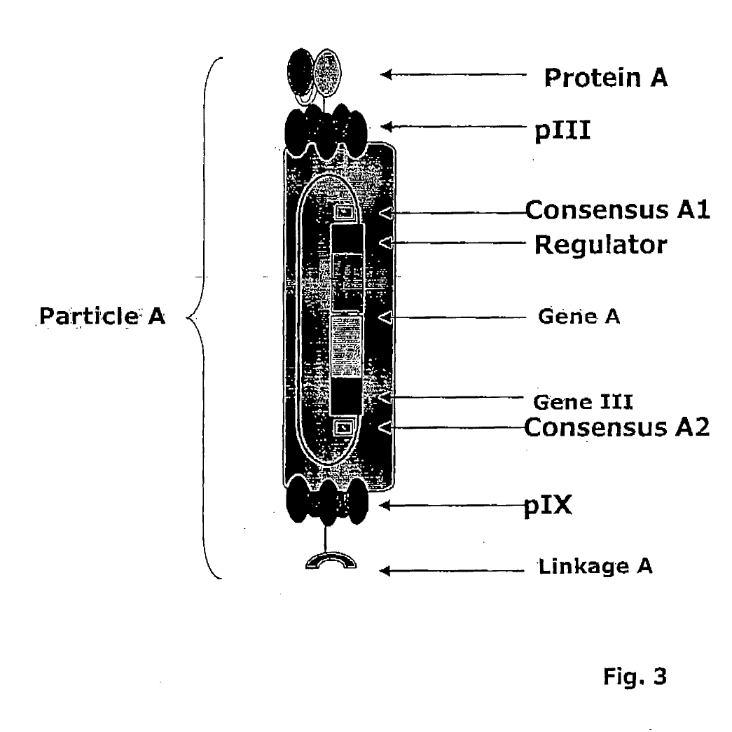

Step 2: Generation of Immobilized Molecule Libraries 2.1 Synthesis of a combinatory molecule library (for an scFv bacteriophage library as an example): According to G. E. Stoica et al., J. Biol. Chem. (2001), a combinatory scFv bacteriophage library (here: with reactivity against pancreas carcinoma) for immobilization on the microchips is established whose individual components (particle A) are characterized as follows (FIG. 3):

At one end of the particle, there is protein A (here: an unknown scFv or Fab of the library to be immobilized) which is covalently and functionally coupled to a surface protein of particle A (here: protein III (pIII) of a filamentous bacteriophage).

The opposing end of the same particle is modified in such a way that a directed coupling of particle A to a coupling area of the microchip is ensured by the activity of linkage A (in this case: streptavidin or a modified streptavidin binding component, or an anti-biotin scFv).

Particle A bears a phagmid with the genetic information for the expression of protein A as a fusion protein with a surface protein (here gene A fused with gene III controlled by the regulator). In addition, there are the attachment sites (consensus A1 and A2) for the specific primers A1 and A2 on the phagmid.

2.2 Coupling of a library: The library constructed according to step 2.1 is applied to the readily prepared microchips, resulting in a binding of the individual components to the coupling areas through the interaction between the particle-A-reactive ligand and linkage A (here: biotin/streptavidin or biotin/anti-biotin scFv). This ensures that there is a high probability that a different unknown particles resides in every coupling area.

Step 3: Generation of the Soluble Molecule Libraries (for a cDNA Expression Library from Primary Tumor Material as an Example)

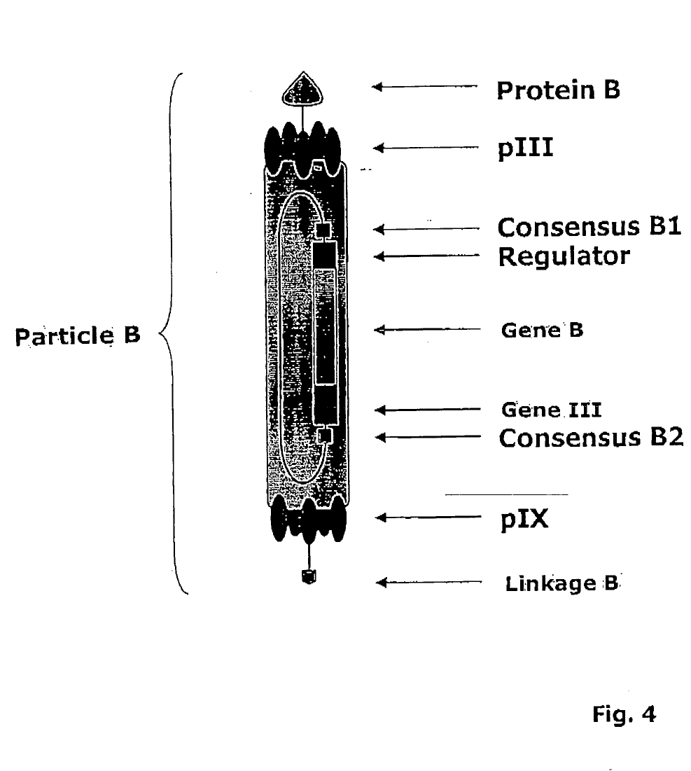

According to X. Cai et al., Proc. Natl. Acad. Sci. USA 92(14): 6531-41 (1995), a soluble cDNA expression library (here: cDNA generated from the mRNA of primary pancreas carcinoma cells) is established whose individual components (particles B) are characterized as follows (FIG. 4):

At one end of the particle, there is protein B (here: an unknown translation product from the primary pancreas carcinoma cells) which is covalently and functionally coupled to a surface protein of particle B (here: protein III (pIII) of a filamentous bacteriophage).

The opposing end of the same particle is modified in such a way that a directed coupling of particle B to a sorting particle through linkage B is ensured (in this case: poly-histidine labeling).

Particle B bears a phagmid with the genetic information for the expression of protein B as a fusion protein with a surface protein (here gene B fused with gene III controlled by the regulator). In addition, there are the attachment sites (consensus B1 and B2) for the specific primers B1 and B2 on the phagmid.

Optionally, particle B can be additionally provided with GFP fusions, e.g., surface protein VIII to enable detection by fluorescence techniques.

Step 4: Detection of the Individual Interactions

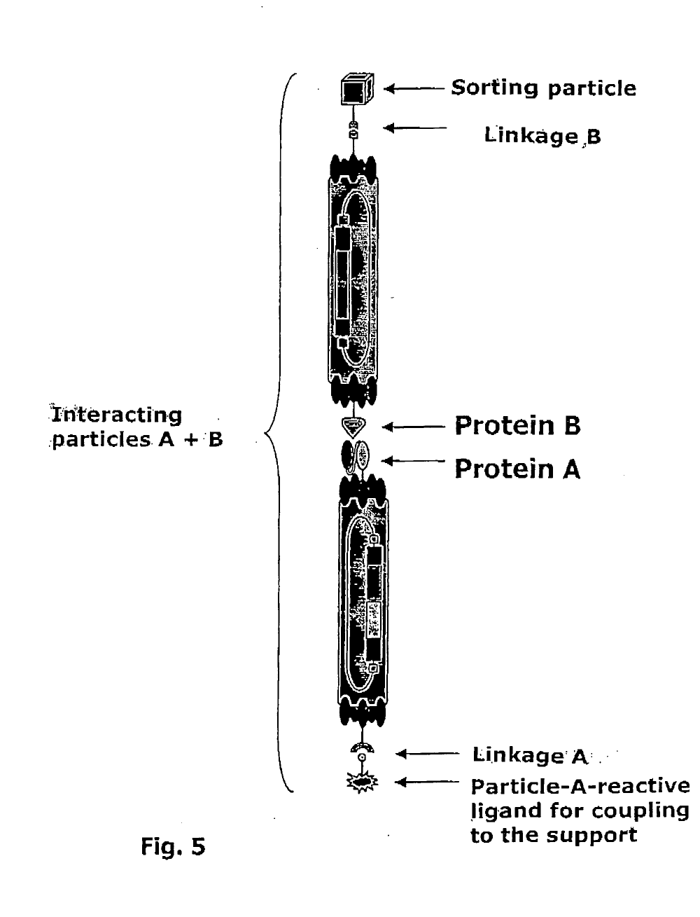

The soluble protein library is added to the immobilized protein library, and non-specific binding activities between the libraries are eliminated by several washing operations. After the coupling to the sorting particle (here: magnetic particle with binding activity for poly-histidine tag, for example, Ni²⁺-NTA or “high graft”), where there is the optional possibility of applying a magnetic field to obtain a vectorial orientation of the interacting particles A+B (see FIG. 5), measurement of the correspondingly interacting particles is effected by, for example, SNOM or two-dimensional scanning fluorescence technique. This results in an assignment of the detected interaction to the coordinates X and Y.

Step 5: Specific Detachment of the Interacting Molecules

The coordinates of the detected interaction are compared with the coordinates of the coupling areas (internal control). The detaching laser (SNOM, alternatively: CW-UV sources) is adjusted onto these coordinates and acts through axis Z on the photolinker layer applied to microchip (FIG. 2), which results in a directed detachment of the interacting particles.

Step 6: First Proof of Principle

According to X. Cai et al., Proc. Natl. Acad. Sci. USA 92(14): 6531-41 (1995), a cDNA mini expression library consisting of CD30 and CEA antigens for immobilization on the microchips is established whose individual components are characterized as described in FIG. 3. At one end of the particle, there are the mentioned antigens which are covalently and functionally coupled to protein III (pIII) of filamentous bacteriophages. The opposing end of the same particle is modified in such a way that a directed coupling of particle A to a biotin coupling area of the microchip is enabled by anti-biotin binding ligands. Each of the two different particles A bears a phagmid with the genetic information for the expression of the different antigens as a fusion with gene III controlled by the regulator. The mini library is applied to the readily prepared microchips, resulting in a binding of the individual components to the coupling areas through the interaction between biotin/anti-biotin ligands. The distribution of the antigen-bearing bacteriophage particles at the coupling areas is directly correlated with the percent proportions of the particles in the mini library.

According to X. Cai et al., Proc. Natl. Acad. Sci. USA 92(14): 6531-41 (1995), a soluble scFv bacteriophage mini library consisting of anti-CD30, anti-CEA and anti-MUC-1 is established whose individual components (particles B) are characterized as follows (FIG. 4): At each end of the particles, there are the scFvs which are covalently and functionally coupled to protein III (pIII) of the filamentous bacteriophages. The opposing end of the same particle is modified in such a way that a directed coupling of particle B to an Ni²⁺-NTA-coupled sorting particle is ensured through the polyhistidine labeling. Each of particles B bears a phagmid with the genetic information for the expression of the scFvs as a fusion with gene III controlled by the regulator.

After the adjustment of different dilutions of CD30 and CEA for the immobilized minilibrary as well as of anti-CD30, anti-CEA and anti-Muc-1 for the soluble minilibrary, mutually interacting particles are detected, detached from the support through the detaching laser, collected and bound to appropriate columns through the coupled magnetic sorting particle. After intensive washing, the magnetically bound particles are eluted any analyzed by polymerase chain reaction, or the binding to CD30+ and CEA+cells and membrane fractions is confirmed by in-vitro binding studies (ELISA, flow cytometry) of the scFv-bearing bacteriophages.

Step 7: Collecting the Interacting Molecules

The intercoupled particles A+B are directed through the sorting particle (here: magnetic particle with binding activity for the poly-histidine tag, e.g., through metal chelates or “high graft”) via microfluid systems into the final microreaction vessels.

Step 8: Analysis of the Interacting Molecules

In the microreaction vessels, the coding regions of the sorted particles A+B are simultaneously amplified by polymerase chain reaction using the specific primers A1/A2 and B1/B2. Aliquots of this reaction mixture can be analyzed by parallel sequencing operations, and thus the sequences of the genes A+B can be simultaneously established and the encoded protein sequences derived therefrom by standard methods.

The activity of the available translation products is characterized after in vitro and in vivo expression in known systems

1. A method for the selection of unknown molecules comprising the following steps:
(a) immobilizing molecules of a combinatory molecule library on a support, wherein each individual molecule is immobilized in a coupling-reactive area having dimensions of smaller than 50 nm×50 nm;
(b) attaching complementary singular binding molecules or cells to a particular immobilized molecule;
(c) selective detaching the mutually interacting singular molecules from the support without disrupting the binding between the molecules of the combinatory molecule library and the complementary singular binding molecules or cells; and
(d) analyzing the material detached in step (c).

2. The method according to claim 1, wherein said support is made of a signal-transmissive material, especially a material transmissive for light, heat and/or magnetic fields, more preferably a light-transmissive material.

3. The method according to claim 1, wherein each individual molecule is immobilized in a coupling-reactive area having dimensions of smaller than 10 nm×10 nm, and/or the distance between the coupling-reactive areas is from 100 nm to 100 μm.

4. The method according to claim 3, wherein step (a) comprises the application of an anchor group reactive towards light, heat or magnetism, especially photolabile linkers, such as o-nitrobenzyl ester, pivaloyl or triazene compounds, or a thermosensitive polymer to the support and the adjustment of said coupling-reactive areas.

5. The method according to claim 1, wherein said soluble molecule library is a cDNA expression library for binding proteins, a cDNA expression library of a cell, or a synthetic peptide or molecule library, and said molecules are, in particular, peptides, proteins, conjugated ribosomes or conjugated bacteriophages.

6. The method according to claim 1, wherein said complementary molecule library is selected from peptides, proteins, conjugated ribosomes, conjugated bacteriophages, liposomes, labeled particles.

7. The method according to claim 1, wherein one or more washing operations are effected after step (b).

8. The method according to claim 1, in which the analysis of the position of the molecule, cell or molecule-cell interaction is effected by an optical, magnetic or profilometric measuring method.

9. The method according to claim 1, wherein a combinatory library is immobilized on a transparent support, and this immobilization layer can be selectively detached by a photonic or thermal interaction.

10. The method according to claim 1, wherein step (d) is effected by MALDI (matrix-associated laser desorption/ionization) or ESI (electrospray ionization) with subsequent mass analysis, SPR (surface plasmon resonance), multispectral analysis, fluorescence correlation spectroscopy (FCS), or polymerase chain reaction (PCR).

11. A biochip for the selection of unknown single molecules comprising a support as defined in claim 1 which is suitable for immobilizing or has been immobilized with a combinatory molecule library, to which support has been applied an anchoring group as defined in claim 4 and on which a combinatory molecule library can be or has been immobilized, ensuring that precisely one molecule can be or has been taken up on the support per coupling area.

12. A method for the preparation of a biochip according to claim 11, comprising:
(i) producing coupling areas on a support which are suitable for taking up precisely one molecule; and/or
(ii) immobilizing molecules from a combinatory molecule library on a support.

13. A device for the automated measurement of molecular or cellular interactions and their coordinative assignment with a spatial resolution of <100 nm, especially for use in a method according to claim 1 and/or for reading out a biochip according to claim 11.

14. The device according to claim 13 which is suitable for sorting the interacting molecules.

15. The device according to claim 13 which is suitable for analyzing the interacting molecules, preferably identifying the detected molecule by MALDI (matrix-associated laser desorption/ionization) or ESI (electrospray ionization), SPR, multispectral analysis, FCS or PCR.
